(19) United States
(12) Patent Application Publication
(10) Pub. No.: US 2012/0111799 A1
(43) Pub. Date: May 10, 2012

(54) INSTALLATION AND A METHOD FOR MONITORING THE QUALITY OF WATER IN A POTABLE WATER NETWORK

(75) Inventors: Cyrille Lemoine, Sartrouville (FR); Marc Moreau, Suresnes (FR)

(73) Assignee: VEOLIA EAU - COMPAGNIE GENERALE DES EAUX, Paris (FR)

(21) Appl. No.: 13/258,995

(22) PCT Filed: Mar. 19, 2010
(86) PCT No.: PCT/FR2010/050497

§ 371 Date: Oct. 26, 2011

(30) Foreign Application Priority Data
Mar. 24, 2009 (FR) 0951859

(51) Int. Cl. E03B 7/07; C02F 1/32; G01N 33/18; C02F 1/72

(52) U.S. Cl. 210/739; 210/96.1; 210/87

(57) ABSTRACT

The invention relates to an installation (30) for monitoring the quality of water and designed to be installed on a pipe (18) dedicated to distributing water to a consumer. The invention is characterized in that the installation comprises:
a probe (32) for measuring at least one parameter of the water flowing in the pipe;
a contamination detector device (46) for detecting the presence, if any, of contamination on the basis of measurements performed by the probe;
a reverse flow detector device (36) for detecting reverse flow in said pipe;
a disinfection device (40) for disinfecting the water in the pipe and/or a cutoff valve for closing the pipe; and
a control member (50) for actuating the disinfection device and/or the cutoff valve as a function of data provided by the contamination detector device and the reverse flow detector device, whereby the quality of the water delivered to or returned by the consumer is monitored.

The present invention relates to the field of water quality control in potable water distribution networks.

At present, although the quality of water in networks is under overall control, spot contamination or pollution can nevertheless appear as a result of a pipe rupturing or breaking, or because of water being returned, or indeed because of deliberate intrusions of contaminants into the potable water network.

The term “return of water” is used to mean a flow of water flowing in abnormal manner from a consumer to the potable water network.

Certain large consumers of water, such as, for example: hospitals, creches, retirement homes, restaurants, and more generally establishments receiving the public, are particularly sensitive to risks of contamination.

In order to monitor the quality of water in the network, document WO 2007/011352 proposes filtering and purifying water at the point of delivery (close to the consumer) using filter and purification cartridges.

The drawback of that system is that it requires the cartridges to be changed regularly, which can be expensive and inconvenient. Furthermore, in the event of severe contamination, the use of a filter cartridge may be found to be insufficient for blocking the contamination.

Document U.S. Pat. No. 6,245,224 describes an installation for monitoring water quality, but it does not take return of water into consideration. Furthermore, that installation is very complex insofar as it requires a plurality of quality sensors in the network, flow rate sensors, a bidirectional communications system, and a central system for analyzing data.

An object of the present invention is to provide a monitoring installation for monitoring water quality and designed to be connected to a pipe dedicated to delivering water to a consumer, which installation seeks to mitigate the above-mentioned drawbacks.

The invention achieves this object by the fact that the installation comprises:
a probe for measuring at least one parameter of the water flowing in the pipe;
a contamination detector device for detecting the presence, if any, of contamination on the basis of measurements performed by the probe;
a reverse flow detector device for detecting reverse flow in said pipe;
a disinfection device for disinfecting the water in the pipe and/or a cutoff valve for closing the pipe; and
a control member for actuating the disinfection device and/or the cutoff valve as a function of data provided by the contamination detector device and the reverse flow detector device, whereby the quality of the water delivered to or returned by the consumer is monitored.

Thus, by means of the invention, the disinfection device is activated only when necessary, in other words only when contamination is detected. The energy required for causing the disinfection device to operate is thus minimized.

In addition, in the event of severe contamination, the cutoff valve is actuated so as to close the pipe and protect the consumer if the contamination is coming from the distribution network, or so as to protect the network if the contamination is coming from the consumer.

Furthermore, management of returns of water is improved compared with the prior art insofar as the safety device, i.e. the disinfection device and/or the cutoff valve, are preferably actuated solely when a return of water is detected by the reverse flow detector device simultaneously with contamination being detected.

Another advantage of the installation is that it is independent. Furthermore, unlike the prior art, the installation of the invention enables the consumer to be made safe locally as soon as the entire installation has been fitted to a pipe connecting the consumer to the remainder of the network.

Preferably, the invention further includes a consumption meter comprising said reverse flow detector device.

The consumption meter is the equipment that measures the volume of water consumed by the consumer. Meters are read periodically by an operator or automatically by remote-reading.

Advantageously, the installation further includes a warning device for warning the consumer and designed to be activated as a function of data supplied by the contamination detector device and by the reverse flow detector device.

Thus, in the event of contamination being detected, or indeed in the event of a return of water being detected in association with contamination being detected, a warning is triggered in order to warn the consumer.

In an advantageous embodiment, the installation further includes a transmission unit for transmitting, to a control center, information picked up by the contamination detector device and the reverse flow detector device, together with the state of the disinfection device and/or the state of the valve.

The control center collects information coming from all of the installations of the potable water network. Warnings are thus also transmitted to the control center. Transmission preferably takes place using wireless transmission device.

Where appropriate, the control center is capable of drawing up a map of the contamination and possibly of closing one or more pipes of the network in order to provide consumer safety.

Preferably, said parameter measured by the probe is taken at least from: chlorine concentration, conductivity, temperature, turbidity, and organic matter concentration.

Also preferably, the probe is of the multi-sensor type in the sense that it measures a plurality of parameters.

Furthermore, the probe advantageously takes measurements without using any reagent and without rejecting water to the outside of the pipe.

In other words, measurements are taken without wasting water.

Advantageously, and unlike the prior art, the disinfection device of the invention does not use any consumables (such as filter cartridges), or any reagents, whereby the installation of the invention presents an operating cost and an environmental footprint that are small relative to those of the prior art.

Advantageously, the disinfection device comprises an electrochemical system generating oxidizing compounds, e.g. of the type comprising chlorine in its various forms, or indeed peroxides, percarbonates, or indeed ozone.

The oxidizing compounds are thus generated on site, thus making it possible to avoid storing chemicals on consumer premises.

Advantageously, the disinfection device comprises an ultraviolet lamp that may optionally be associated with the above-mentioned electrochemical system.

In a particularly advantageous aspect of the invention, the probe measures at least two parameters, and the contamination detector device comprises a device for performing correlated analysis of said at least two parameters.

The principle of performing correlated analysis of a plurality of parameters is explained in document FR 2 911 960. It is based on cross analysis of a set of indicators that are not pertinent on their own but that, when taken together, can make it possible to recognize a quality problem that justifies taking safety measures, even without the nature of the problem being identified exactly. By means of that algorithm, it is possible to distinguish natural variability in the measured parameters from accidental variability, and consequently to avoid false alerts. Naturally, and without going beyond the ambit of the present invention, the contamination detection system may be based on any other algorithm, for example of the trigger threshold type.

The invention also provides a distribution network including a plurality of pipes for distributing water to consumers, together with at least one installation of the invention, said installation being mounted on one of said pipes.

The installation is preferably mounted on a connection pipe, i.e. a pipe connecting a delivery point (the consumer) to a main pipe of the network.

Advantageously, the distribution network of the invention further includes a control center that receives the information supplied by the contamination detector device and the reverse flow detector device, together with the state of the disinfection device and/or the state of the valve, the control center also including a device for remotely controlling the control member of the installation.

Thus, in the event of contamination being detected with one or more consumers, the control center is suitable for actuating the cutoff valve and/or for activating the disinfection device in any monitoring installation of the network. As a result, in the event of contamination being detected with a consumer, the operator of the control center may for example activate the safety device of consumers neighboring the consumer where the contamination has been detected.

Advantageously, the control center further includes a hydraulic model and/or a quality model that receives the data supplied by the contamination detector device in such a manner as to predict the propagation in the network of detected contamination, if any.

Thus, the present invention enables the distribution network to be monitored overall. Predicting how contamination will diffuse makes it possible to take targeted measures quickly for the purpose of preserving the safety of consumers.

Finally, the invention provides a method of controlling the quality of the water in a distribution network that includes at least one monitoring installation of the invention, in which method the disinfection device and/or the cutoff valve is actuated when a reverse flow is detected and/or when contamination is detected.

As mentioned above, the cutoff valve is preferably, but not exclusively, activated when both contamination and a return of water are detected simultaneously.

Preferably, a warning is triggered if the disinfection device and/or the cutoff valve is/are actuated.

The invention can be better understood and its advantages appear better on reading the following detailed description of an embodiment given by way of non-limiting example. The description refers to the following accompanying figures:

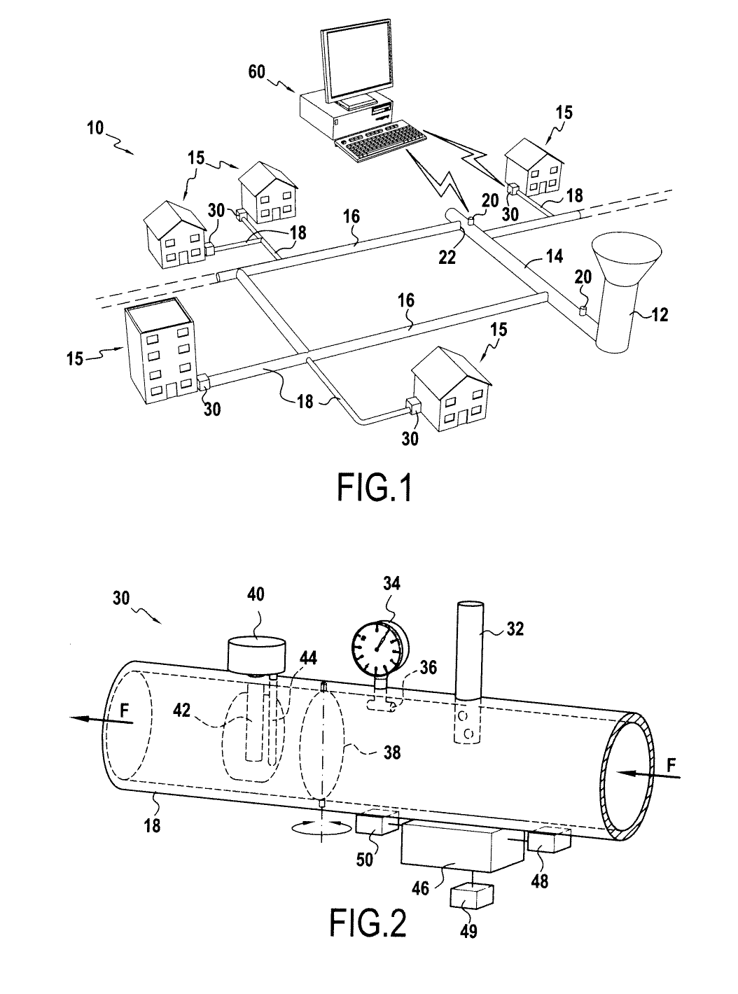

FIG. 1 shows a portion of a potable water distribution network comprising a plurality of monitoring installations of the invention; and

FIG. 2 shows an embodiment of a monitoring installation of the invention.

The potable water distribution network 10 shown in FIG. 1 corresponds to the downstream portion of a main network fed by one or more waterworks (not shown herein).

The network 10 is fed by a reservoir 12, specifically a water tower, itself connected to the main network via pipes that are not shown herein.

Conventionally, potable water is taken from the reservoir 12 by transfer pipes 14 that are dedicated to conveying water over long distances. A plurality of distribution pipes 16 are connected to the transfer pipes 14, and each consumer 15, or subscriber, is connected to the distribution pipes 16 via connection pipes 18. The term “consumer” is used to mean an individual person or a group of people who consume water, for example a hospital, a crèche, an apartment block, or a plurality of individual houses connected to the same connection pipe. It can be understood that under most circumstances, the diameter of a connection pipe 18 is significantly smaller than the diameters of the transfer and distribution pipes 14 and 16.

The network 10 is also preferably fitted with sensors 20, i.e. hydraulic sensors of the pressure sensor or flow rate sensor type, and also with sensors for measuring the concentration of certain chemical species, such as chlorine, for example.

In the example of FIG. 1, the sensors 20 are mounted at one of the nodes 22 of the network 10, and at the immediate outlet from the reservoir 12. Naturally, the number of sensors shown is not limiting and a greater number of sensors may be provided and they may be provided at other locations.

In accordance with the invention, the distribution network 10 has a plurality of monitoring installations 30 that are mounted specifically on the connection pipes 18. In the invention, a monitoring installation 30, as can be seen more clearly in FIG. 2, includes a multi-sensor type probe 32 that is installed on the connection pipe, at the upstream end of the installation relative to the normal flow direction F along the pipe 18.

The probe 32 performs measurements without rejecting water to outside the pipe 18 and it does not require reagents to be used.

Furthermore, the probe 32 is suitable for measuring a plurality of parameters, namely: chlorine concentration, conductivity, water temperature, turbidity, and also organic matter concentration. Specifically, the probe 32 is preferably made up of a plurality of sensors.

Downstream from the probe 32, the installation 30 further includes a consumption meter 34 that is fitted with a remote-reader device, themselves known. The consumption meter 34 also includes a reverse flow detector device 36. The term “reverse flow” is used to mean a flow of water flowing in the direction opposite to the normal direction F, where the normal direction is from the network to the consumer.

The installation 30 also includes a cutoff valve 38 that, when actuated, serves to close the connection pipe 18.

Furthermore, the installation 30 includes a disinfection device 40 arranged downstream from the meter 34, the cutoff valve 38, and the probe 32. This disinfection device 40 comprises an electrochemical system 42 acting in situ to generate oxidizing compounds in the pipe 18, and preferably also an ultraviolet (UV) lamp 44. In this example, the oxidizing compounds are chlorine in various forms, peroxides, ozone, and hydroxyl radicals. It can thus be understood that when it is activated the disinfection device serves to disinfect the water flowing in the pipe 18.

The installation 30 also includes a contamination detector device 46 that is connected to the probe 32 by the wireless transmission device 48.

The function of the contamination detection device is to detect the presence of contamination, if any, on the basis of measurements performed by the probe 32. Specifically, the detector device implements the correlated analysis algorithm described in document FR 2 911 960.

For example, the simultaneous detection of a drop in the chlorine concentration and a rise in the turbidity associated with a normal water flow going from the network 10 to the consumer 18 indicates that the water is probably contaminated. Such contamination may come from a break or from the detachment of a biofilm covering the inside walls of pipes in the network 10.

In order to actuate the cutoff valve 38 and the disinfection device 40, the monitoring installation 30 also includes a control member 50 connected to the detector device 46 and also to the cutoff valve 38 and to the disinfection device 40.

The control member 50 is preferably connected to the cutoff valve and also to the disinfection device 40 by the transmission device 48.

The function of the control member is thus to control the contamination detector device 46 and the cutoff valve 38, together or separately.

In a first mode of operation, the control member actuates the disinfection device 40 if contamination is detected by the contamination detector device 46. If no return of water is detected by the reverse flow detector device, then the cutoff valve is generally not actuated, unless the detected contamination is particularly severe.

In contrast, and in a second mode of operation, if contamination and a return of water are detected simultaneously, the cutoff valve is actuated so as to close the pipe 18. This prevents the pollutant propagating into the network. In this second mode of operation, the disinfection device may optionally be activated.

For example, a return of water associated with a rise in temperature indicates that the potable water supply network has probably been connected to the consumer's hot water network. This can result in a risk of Legionnella developing on the consumer premises, or even of the distribution network 10 being contaminated if a large amount of water is being returned. According to the invention, the severity of the return of water is determined as a function of the volume of water flowing into the network 10 and as a function of the type of contamination that has been detected.

The monitoring installation 30 also includes a warning device to inform the consumer 15 of the presence of contamination.

According to an advantageous aspect of the invention, the distribution network 10 also includes a control center 60 having the function of controlling the operation of the network 10. To do this, the control center 60 includes a computer processor system serving to model the hydraulic behavior and the quality of the water in the network. The control center 60 thus includes a hydraulic model coupled to a so-called “quality” model, e.g. a model of the rate of decrease in chlorine concentration. It should be specified that such models are already known.

In particular, the control center receives information coming from the various sensors 20 and also from the monitoring installations 30.

In other words, each monitoring installation 30 includes a transmission unit 49 for sending information coming from the contamination detector device 46 and from the reverse flow detector device 36 to the control center 60. The transmission units 49 also send the “open” or “closed” state of the cutoff valve 38 to the control center together with the “active” or “inactive” state of the disinfection device 40.

This information is supplied to the hydraulic and quality models and it makes it possible to supervise the distribution network in overall manner. Specifically, the hydraulic and quality models make it possible, advantageously, to simulate the propagation of contamination detected at some particular point of the network and to trigger preventative action, where necessary. To do this, the control center 60 further includes a device for remotely controlling the control members 50 of the various monitoring installations of the network 10.

Thus, when contamination is detected at one or more consumers 15, the control center 60 is suitable for actuating the cutoff valve 38 of the monitoring installations 30 of the consumers 15 who are situated downstream in the propagation direction as predicted by the above-mentioned models. Other criteria for isolating portions of the network 10 may also be envisaged.

Furthermore, the control center 60 of the network 10 in the invention is suitable for identifying contaminated zones on the basis of information picked up by the contamination detector devices 46.

Furthermore, and preferably, the data transmitted within the monitoring installation 30 and also between the installation 30 and the control center 60 is advantageously encrypted.

1. A monitoring installation for monitoring water quality and designed to be connected to a pipe dedicated to delivering water to a consumer, said installation being characterized in that it comprises:
a probe for measuring at least one parameter of the water flowing in the pipe;
a contamination detector device for detecting the presence, if any, of contamination on the basis of measurements performed by the probe;
a reverse flow detector device for detecting reverse flow in said pipe;
a disinfection device for disinfecting the water in the pipe and/or a cutoff valve for closing the pipe; and
a control member for actuating the disinfection device and/or the cutoff valve as a function of data provided by the contamination detector device and the reverse flow detector device, whereby the quality of the water delivered to or returned by the consumer is monitored.

2. An installation according to claim 1, wherein it further includes a consumption meter comprising said reverse flow detector device.

3. An installation according to claim 1, wherein it further includes a warning device for warning the consumer and designed to be activated as a function of data supplied by the contamination detector device and by the reverse flow detector device.

4. An installation according claims 1, wherein it further includes a transmission unit for transmitting, to a control center, information picked up by the contamination detector device and the reverse flow detector device, together with the state of the disinfection device and/or the state of the valve.

5. An installation according to claim 1, wherein said parameter is taken at least from: chlorine concentration, conductivity, temperature, turbidity, and organic matter concentration.

6. An installation according to claim 1, wherein-the disinfection device comprises an electrochemical system generating oxidizing compounds.

7. An installation according to claim 1, wherein-the disinfection device comprises an ultraviolet lamp.

8. An installation according claim 1, wherein the probe measures at least two parameters, and in that the contamination detector device comprises a device for performing correlated analysis of said at least two parameters.

9. An installation according to claim 1, wherein the probe performs measurements without using any reagent and without rejecting water to the outside of the pipe.

10. A distribution network including a plurality of pipes for distributing water to consumers, and at least one installation according to claim 1, said installation being mounted on at least one of said pipes.

11. A distribution network according to claim 10, wherein it further includes a control center receiving the information supplied by the contamination detector device and the reverse flow detector device, together with the state of the disinfection device and/or the state of the valve, and in that the control center also includes a device for remotely controlling the control member of the installation.

12. A distribution network according to claim 11, wherein the control center further includes a hydraulic model and/or a quality model receiving the data supplied by the contamination detector device in such a manner as to predict the propagation in the network of detected contamination, if any.

13. A method of controlling the quality of water in a distribution network that includes at least one installation according to claim 1, in which method the disinfection device and/or the cutoff valve is actuated when a reverse flow is detected and/or when contamination is detected.

14. A method of controlling water quality according to claim 13, wherein a warning is triggered if the disinfection device and/or the cutoff valve is/are actuated.
